(19) United States
(12) Patent Application Publication
(10) Pub. No.: US 2018/0217155 A1
(43) Pub. Date: Aug. 2, 2018

(54) PROTEOMIC ANALYSIS OF SUBCELLULAR COMPARTMENTS

(71) Applicants: UNIVERSITÉ DE BORDEAUX, Bordeaux (FR); INSERM - INSTITUT NATIONAL DE LA SANTÉ ET DE LA RECHERCHE MÉDICALE, Paris (FR)

(72) Inventors: Frédéric SALTEL, Talence (FR); Anne-Aurélie RAYMOND, Mérignac (FR); Zakaria EZZOUKHRY, Mérignac (FR)

(21) Appl. No.: 15/745,261

(22) PCT Filed: Jul. 13, 2016
(86) PCT No.: PCT/FR2016/051808

§ 371 Date: Jan. 16, 2018

(30) Foreign Application Priority Data
Jul. 15, 2015 (FR) 1501491

(51) Int. Cl. G01N 33/68; G01N 33/58; G01N 1/28

(52) U.S. Cl. CPC G01N 33/6842; G01N 33/6848; G01N 33/582; G01N 2458/15; G01N 2570/00; G01N 2001/284; G01N 1/2813

(57) ABSTRACT

Some embodiments are directed to a method for the subcellular proteomic analysis of a test biological sample, including metabolic isotopic labelling of proteins of a test biological sample, fixing of the sample, labelling of the test subcellular compartment, laser microdissection of said subcellular compartment, extracting the proteins of said subcellular compartment, reversion of the fixing and proteolysis, analyzing the peptides obtained by mass spectrometry, and identifying the analyzed peptides.

### CROSS REFERENCE TO RELATED APPLICATIONS

This application is a national phase filing under 35 C.F.R. § 371 of and claims priority to PCT Patent Application No. PCT/FR2016/051808, filed on Jul. 13, 2016, which claims the priority benefit under 35 U.S.C. § 119 of French Patent Application No. 1501491, filed on Jul. 15, 2015, the contents of each of which are hereby incorporated in their entireties by reference.

### BACKGROUND

Some embodiments relate to a novel process for the proteomic analysis of subcellular compartments, combining laser microdissection followed by analysis by mass spectrometry.

Some embodiments are especially applicable in the field of research but also in the field of medical diagnostics.

In the description below, the references between square brackets ([ ]) refer to the list of references presented at the end of the text.

Analysis by mass spectrometry makes it possible to identify and quantify the proteins from a biological sample. This analysis may be performed from whole tissues or from cell extracts and after biochemical separation of cellular compartments within the context of a subcellular proteomic analysis. However, the specificity and reproducibility of the methods for subcellular fractionation are widely debated, due to the biases induced by cell lysis, which leads to destabilization of the cellular compartments and the molecular complexes from which they are composed [1, 2].

Currently, MALDI imaging (Matrix-Assisted Laser Desorption/Ionization Imaging Mass Spectrometry or MALDI-IMS) brings a new dimension to histological analyses, since it combines sensitivity and selectivity and makes it possible to directly visualize the arrangement of biomolecules in a tissue. However, at present, this new technology has two significant limitations: (i) a best-case lateral resolution of 20 μm, which is incompatible with the analysis of subcellular compartments [3], and (ii) unlike the bottom-up approach based on the digestion of the proteins, MALDI imaging requires a top-down approach which relies on the analysis of whole proteins and which is still currently in its infancy in this respect [4-6]. Gregorich et al. ([4]) clearly highlight the obstacles to implementing this technology in terms of solubility of the proteins, separation and detection of large proteins, coupled with a decreased yield and a lack of automation. Within the context of their work using the pairing of MALDI imaging and top-down identification with mass spectrometry, Ye et al. ([5]) highlight the limits of this imaging technique, which requires working with frozen sections and does not make it possible to work with fixed sections, and the limited resolution of which does not enable subcellular analysis. Finally, Ait-Belkacem et al. ([6]), who use the pairing of MALDI imaging and top-down identification with mass spectrometry to characterize the molecular structures of glioblastomas, in this case only obtain a spatial resolution of 30 μm, which does not enable analysis on the subcellular scale.

Document EP1601450 ([9]), which describes a method for detecting analytes in a lyzate, also does not enable analysis on a subcellular scale.

Leverenz et al. ([10]) describe a method of subcellular proteomic analysis of a brain sample including a step of laser microdissection of Lewy bodies followed by a step of analysis by mass spectrometry. However, the level of resolution is unsatisfactory for real analysis on the subcellular scale.

Document US 2011/0215233 ([11]) describes a process for subcellular analysis by laser ablation and mass spectrometry. However, although a nucleus/cytoplasm separation is carried out, and the authors mention the possibility of applying their process to cellular organelles, no examples are provided at this level of resolution. Thus, the level of resolution does not appear to be sufficiently satisfactory for real analysis on the subcellular scale.

### SUMMARY

It may therefore be beneficial to provide an alternative approach which addresses or overcomes these drawbacks, disadvantages and obstacles of the related art, in particular for a process which makes it possible to obtain a better resolution, but also to enhance or improve the specificity and reproducibility of the analyses.

To this end, the inventors have developed a novel approach which represents an alternative to several methodologies, such as cellular compartmentalization by centrifugation and in certain cases immunoprecipitation. Moreover, given current advances and spatial limitations and limitations in terms of identification, this novel approach proposes an alternative to MALDI imaging.

Some embodiments are directed to a process enabling the identification and quantification of the proteins present in a cellular compartment (endoplasmic reticulum, centriole, Golgi apparatus, synapses, etc.). This analysis cannot be performed by related art or conventional techniques (mass spectrometry, MALDI-IMS) due to the non-specificity and non-reproducibility of the methods for subcellular fractionation, on the one hand, and the significant contamination by external elements (ambient air, solutions, etc.) on the other hand, and also the technical limiting of these approaches to the micrometer scale.

The process of some embodiments combines a step of isotopic labeling of a test biological sample (e.g. a cell culture), a step of laser microdissection for the selective cutting out of subcellular elements, and a proteomic analysis by mass spectrometry (cf. FIG. 1).

This novel approach, applicable to human or animal cell cultures or to animal tissue sections, combines laser microdissection which enables selective cutting out of the subcellular elements (at a resolution of 0.6 μm) and analysis by mass spectrometry. This novel approach thereby makes it possible to work in a spatial resolution which is compatible with the separation of subcellular compartments such as the nucleus, cytoplasmic membrane, nuclear membrane, vesicles, mitochondria, lysosome, centriole, proteasome, focal adhesions, lamellipodium, filopodia, invadosome rosette, endoplasmic reticulum or Golgi apparatus. It is also possible to cut out extracellular compartments present between two cells, such as cell-cell junctions (e.g. synapses). In addition, this original, novel combination makes it possible to improve the specificity and reproducibility of the analyses compared to the methods currently employed of subcellular fractionation followed by analysis by mass spectrometry.

This novel approach is of interest first and foremost to research laboratories, many of which are seeking novel methods to identify new partners within organelles or subcellular structures. In addition, this novel approach makes it possible to compare the protein composition of a subcellular compartment across different cell samples, for example the composition of a subcellular compartment originating from a normal cell and that of a subcellular compartment originating from a tumor cell. This novel approach also makes it possible to identify proteins which are overexpressed in the context of different pathological conditions. Finally, being able to target a subcellular compartment makes it possible to identify and analyze proteins which, with a non-targeted approach, would be in a minority and not detectable within a much more complex range of proteins.

Thus, some embodiments are directed to a process for the subcellular proteomic analysis of a test biological sample, including a step of laser microdissection of a subcellular compartment of the sample, followed by a step of analysis by mass spectrometry of the constituent elements of the subcellular compartment.

“Test biological sample” means, for example, a sample of human or animal cells or a sample resulting from an animal tissue section, for which sample it is desired to know the qualitative and/or quantitative protein(s) composition of at least one subcellular compartment.

“Constituent elements of the subcellular compartment” means any molecule or protein present within a microdissected compartment. By way of example, filamentous actin is a constituent element of invadosome rosettes, DNA is a constituent element of the nucleus, etc.

According to a particular embodiment of the present invention, the process for proteomic analysis may also include, prior to the microdissection step, a step of metabolic isotopic labeling of the proteins from the test cell sample, a step of fixation of the cells (e.g. in order to limit the Z contaminants in experiments on whole cells, it is possible beforehand to fix and enclose the cells in paraffin and produce 3 μm sections) of the sample and/or a step of labeling the test subcellular compartment.

According to a particular embodiment of the present invention, the process for proteomic analysis may also include, subsequently to the step of laser microdissection and prior to the step of analysis by mass spectrometry, a step of extraction of the proteins of interest from the subcellular compartment, of reversion of the fixation and/or of proteolysis.

According to a particular embodiment of the present invention, the process for subcellular proteomic analysis includes, or consists of, the following steps:
a) fixation of the sample;
b) labeling of the test subcellular compartment;
c) laser microdissection of the subcellular compartment;
d) extraction of the proteins from the subcellular compartment, reversion of the fixation, and proteolysis;
e) analysis of the peptides resulting from step d) by mass spectrometry;
f) identification of the peptides analyzed.

According to another particular embodiment of the present invention, the process for subcellular proteomic analysis includes, or consists of, the following steps:
a) metabolic isotopic labeling of the proteins from a test biological sample;
b) fixation of the sample;
c) labeling of the test subcellular compartment;
d) laser microdissection of the subcellular compartment;
e) extraction of the proteins from the subcellular compartment, reversion of the fixation, and proteolysis;
f) analysis of the peptides resulting from step e) by mass spectrometry;
g) identification of the peptides analyzed.

The step a) of isotopic labeling may be carried out by any labeling technique known to those of ordinary skill in the art. It should be noted that metabolic isotopic labeling, of SILAC type, is not performed when the test biological sample is an already-labeled tissue section. In this case, the process of the invention, and especially the step of laser microdissection, may be automated (cf. FIG. 3).

Carrying out at least a portion of the steps of the process may advantageously be automated. This may especially be the step of laser microdissection. In particular, in the case in which the process does not include the isotopic labeling step, this automation relates to the step of laser microdissection. This automation advantageously makes it possible to obtain a sufficient amount of material for analysis by mass spectrometry and the identification of the peptides.

According to a particular embodiment of the present invention, the step of fixation is performed with a crosslinking agent, for example paraformaldehyde, formalin or glutaraldehyde.

According to a particular embodiment of the present invention, the subcellular compartment is chosen from the group including or consisting of the nucleus, cytoplasmic membrane, nuclear membrane, vesicles, mitochondria, lysosome, centriole, proteasome, focal adhesions, lamellipodium, filopodia, invadosome rosette, endoplasmic reticulum, Golgi apparatus, and cell-cell junctions (e.g. synapses).

The step c) of labeling, hence of pinpointing the subcellular compartment of interest, may be performed by different peptides or drugs. According to a particular embodiment of the present invention, the step of labeling the subcellular compartment is performed with a fluorescent marker. This labeling may be performed after the fixation step, or before by constitutive expression, in the subcellular compartment of the test sample, of a marker, typically a fluorescent marker. For example, this is the Lifeact peptide or phalloidin for the filamentous actin of invadosome rosettes, but also DAPI as DNA intercalator which makes it possible to visualize nuclei, ER tracker for the endoplasmic reticulum, etc. Alternatively or additionally, this marker may be expressed by the cells in which the laser microdissection of a subcellular compartment is carried out.

The step e) of extraction of the proteins from the test subcellular compartment, of reversion of the fixation and/or of proteolysis may be performed by any techniques known to those of ordinary skill in the art.

Some other embodiments are directed to a process for the in vitro identification of constituent elements from a biological sample from a subject, including a step of laser microdissection of a subcellular compartment of the sample followed by a step of analysis by mass spectrometry of the constituent elements of the subcellular compartment, and of qualitative and/or quantitative comparison of the constituent elements analyzed relative to a reference sample.

The constituent element may for example be a protein, especially a protein present in the invasion complex, referred to as invadosome. Invadosomes, which include podosomes in normal cells and invadopodia in tumor cells, form actin-rich protein complexes which are specialized in the proteolytic degradation of the extracellular matrix. This proteolytic activity gives tumor cells the ability to cross anatomical barriers and to migrate through tissues.

According to a particular embodiment of the present invention, the process for in vitro identification also includes, prior to the step of microdissection, at least one step chosen from the group including or consisting of: metabolic isotopic labeling of the proteins from the test biological sample, fixation of the sample, and labeling of the test subcellular compartment.

According to a particular embodiment of the present invention, the process for in vitro identification also includes, subsequently to the step of laser microdissection and prior to the step of analysis by mass spectrometry, at least one step chosen from the group including or consisting of: extraction of the proteins of interest from the subcellular compartment, reversion of the fixation, and proteolysis.

According to a particular embodiment of the present invention, the process for in vitro identification includes, or consists of, the following steps:
a) fixation of the sample;
b) labeling of the test subcellular compartment;
c) laser microdissection of the subcellular compartment;
d) extraction of the proteins from the subcellular compartment, reversion of the fixation, and proteolysis;
e) analysis of the peptides resulting from step d) by mass spectrometry;
f) identification of the peptides analyzed;
g) qualitative and/or quantitative comparison of the peptides resulting from step f) relative to the peptides present in a reference sample or to a reference value.

According to another particular embodiment of the present invention, the process for in vitro identification includes, or consists of, the following steps:
a) metabolic isotopic labeling of the proteins from a biological sample from a subject suffering from a tumor;
b) fixation of the sample;
c) labeling of the test subcellular compartment;
d) laser microdissection of the subcellular compartment;
e) extraction of the proteins from the subcellular compartment, reversion of the fixation, and proteolysis;
f) analysis of the peptides resulting from step e) by mass spectrometry;
g) identification of the peptides analyzed;
h) qualitative and/or quantitative comparison of the peptides resulting from step g) relative to the peptides present in a reference sample or to a reference value.

According to a particular embodiment of the invention, the subcellular compartment includes or consists of invadosome rosettes.

The step a) of isotopic labeling may be carried out by any labeling technique known to those of ordinary skill in the art. According to a particular embodiment of the present invention, the step of isotopic labeling is performed by the SILAC method (Stable Isotope Labeling by Amino acids in Cell culture). This SILAC method includes or consists of replacing one or more amino acid(s) from the proteins of the test sample with their heavy-isotope equivalent.

Applied to cells in culture, SILAC labeling is a metabolic labeling of cells grown in a culture medium containing one or more amino acid(s) labeled with one or more heavy-isotope(s) (for example ¹³C and/or ¹⁵N). The heavy amino acids are incorporated during cell doubling to give rise to total labeling of the cell proteome. The SILAC method has the advantage of early incorporation of the isotopes into living cells in culture, thereby enabling homogeneous labeling of the proteins. This method was initially conceived to be able to mix, according to a 1:1 ratio from the time of harvesting, cells from two experimental conditions to be compared and thereby to limit technical biases which may be introduced by uneven losses of proteins during the steps of cell lysis and other treatments of the samples (enrichment, desalting, etc.).

The SILAC strategy may also be applied to whole animals, and especially rodents. The latter are fed with feed containing the heavy-isotope amino acids. The tissues recovered from these animals thus contain proteins labeled with heavy isotopes. Advantageously, the SILAC method may enable the incorporation of isotope(s) having a sufficient difference in mass to discriminate between a heavy peptide and a non-heavy peptide. Moreover, the SILAC approach enables complete and stable labeling of the whole of the cell proteome, thereby ensuring quantification by mass spectrometry with very good reproducibility and repeatability.

According to a particular embodiment of the present invention, the step of fixation is performed with a crosslinking agent, for example paraformaldehyde, formalin or glutaraldehyde.

According to a particular embodiment of the present invention, the subcellular compartment is chosen from the group including or consisting of the nucleus, cytoplasmic membrane, nuclear membrane, vesicles, mitochondria, lysosome, centriole, proteasome, focal adhesions, lamellipodium, filopodia, invadosome rosette, endoplasmic reticulum, Golgi apparatus, and cell-cell junctions (e.g. synapses).

The step c) of labeling, hence of pinpointing the subcellular compartment of interest, may be performed by different ways for labeling the subcellular compartment, such as peptides or drugs. According to a particular embodiment of the present invention, the step of labeling the subcellular compartment is performed with a fluorescent marker. This labeling may be performed after the fixation step, or before by constitutive expression, in the subcellular compartment of the test sample, of a marker, typically a fluorescent marker. For example, this is the Lifeact peptide or phalloidin for the filamentous actin of invadosome rosettes, but also DAPI as DNA intercalator which makes it possible to visualize nuclei, ER tracker for the endoplasmic reticulum, etc.

The step e) of extraction of the proteins from the test subcellular compartment, of reversion of the fixation and/or of proteolysis may be performed by any techniques known to those of ordinary skill in the art.

“Reference sample” of step h) means any sample in which the concentration of peptides, after comparison with the concentration of peptides of the test cell sample, offers an indication as to the invasive capacity of a tumor in the subject from whom the test cell sample originates.

By way of examples of reference samples of step h), mention may be made of cell samples originating from a healthy individual or originating from an individual suffering from a tumor, or else a protein solution at a determined concentration.

“Healthy individual” means an individual who does not have any pathological conditions, in particular an individual who does not have a tumor.

Some other embodiments relate to a kit for the subcellular proteomic analysis of a test biological sample, especially for performing the process for subcellular proteomic analysis of the invention, including:
at least one device for labeling the test subcellular compartment as defined above,
at least one device for reversion of the fixation and one device for carrying out the proteolysis, and
at least one reference protein solution at a determined concentration.

The kit according to some embodiments also preferably or possibly includes at least one of the following devices, preferably or possibly all of the following devices:
a device for isotopically labeling the proteins from the test sample,
a device for fixing the cells of the sample, and
a device for extracting the proteins from the subcellular compartment.

The kit of some embodiments may also include all the devices necessary to perform the process for subcellular proteomic analysis of the invention.

Other advantages may also become apparent to those of ordinary skill in the art on reading the examples below, illustrated by the appended figures and given by way of illustration.

### BRIEF DESCRIPTION OF THE FIGURES

FIG. 1 represents the main steps of an embodiment of the process of the invention.

FIG. 2 represents (A-C) a fluorescence micrograph of NIH-3T3-Src cells labeled with DAPI and phalloidin in order to reveal, respectively, nuclear DNA and filamentous actin (the invadosome rosettes are indicated by arrows). (D-E). These micrographs show the same cell type stably expressing the peptide Lifeact-RFP; the microdissected elements are outlined by dotted lines. (F) This diagram shows a rosette outlined manually by the experimenter and cut out by a first laser. (G-H) This diagram represents the process of recovering the microdissected elements by a second laser which catapults the cut-out rosette to the collector cap. (I) This micrograph shows the fluorescence of the microdissected elements in the collector cap; the associated diagram represents this same collector cap with the microdissected and recovered elements.

FIG. 3 represents (A) the procedure for automation of the step of laser microdissection, (B) a representative fluorescence micrograph showing the rosettes which were selected by the automation process and which were cut out by laser microdissection.

FIG. 4 represents (A) an analysis by mass spectrometry carried out on a range of amounts of proteins extracted from total NIH-3T3-Src cells labeled according to the SILAC method with a heavy isotope (^{C13}R and ^{C13}K). For each amount of proteins injected there is a corresponding sum of the intensities of all the labeled peptides detected by mass spectrometry. The sum of the intensities of all the labeled peptides resulting from 40 000 rosettes cut out by laser microdissection was compared to this range, to deduce therefrom an amount of 72 ng of proteins extracted (table, cf. line indicated with an arrow, and corresponding graphical representation below indicated with an arrow). (B) The comparison of the relative intensities of the peptides identified in the sample of rosettes and the total lyzate sample (100 ng) after standardization over the total sum of intensities detected by MS made it possible to confirm enrichment of the identified proteins in the rosettes (Rosettes/Total ratio of ≥2, grayed-out area indicated with an arrow).

FIG. 5 represents a confocal micrograph of NIH-3T3-src cells constitutively expressing the Lifeact peptide and making it possible to visualize the filamentous actin (A); these cells were transfected with an expression plasmid encoding the fusion protein HA-eEF1A1, the use of an anti-HA antibody making it possible to visualize the localization of this protein (B). The fusion of the labeling of (A) and (B) is represented in (C). The elements within boxes are represented in close-up under each image, respectively.

FIG. 6 represents a confocal micrograph of NIH-3T3-src cells constitutively expressing the Lifeact peptide and making it possible to visualize the filamentous actin (A); these cells were transfected with an expression plasmid encoding the fusion protein HA-Eif2A, the use of an anti-HA antibody making it possible to visualize the localization of this protein (B). The fusion of the labeling of (A) and (B) is represented in (C). The elements within boxes are represented in close-up under each image, respectively.

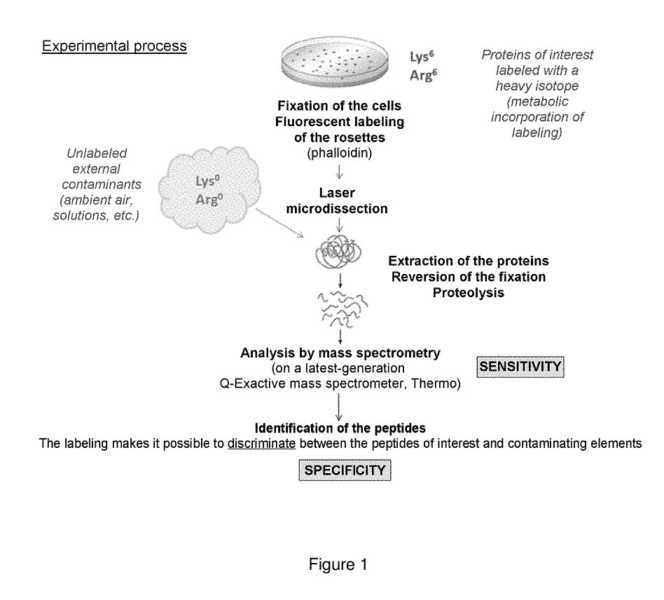

### EXAMPLES OR EMBODIMENTS

### Example 1: Proteomic Analysis of Invadosome Rosettes

While the following protocol can be adapted to other subcellular compartments, the subcellular compartment associated with the capacity of cells to degrade elements of the extracellular matrix was chosen as the model for the present study, namely the invadosome rosette. This type of structure is formed in cells constitutively expressing the active form of the oncogene c-Src, and contributes to the capacity for cellular invasion. It is crucial to know the exact and detailed protein composition of these structures, in order to demonstrate therapeutic targets to attempt to inhibit the invasion of tumor cells. Filamentous actin is the structural and predominant component of these rosettes, and consequently the element which was chosen to pinpoint these rosettes at the time of the laser microdissection.

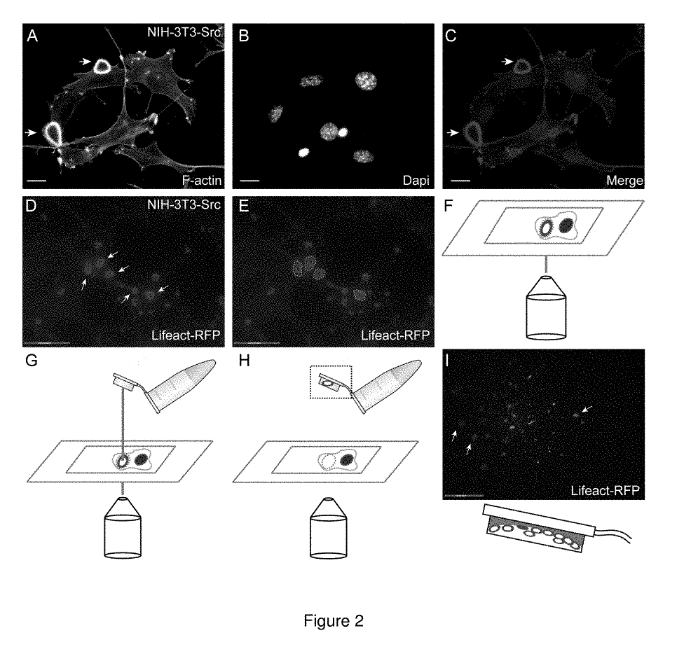

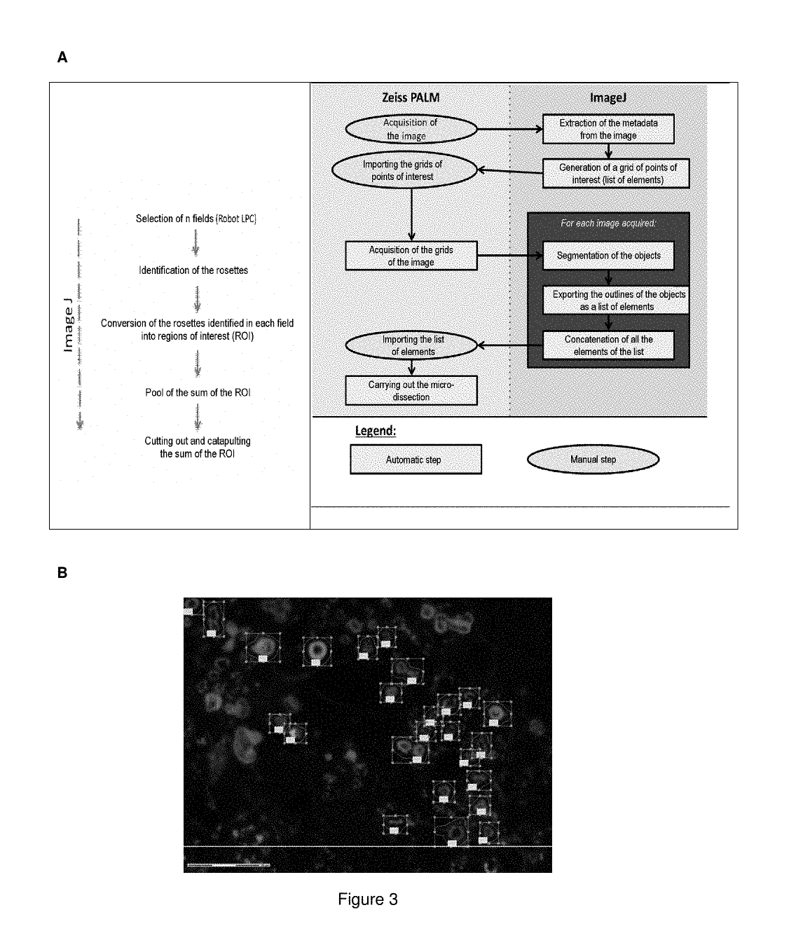

The experiments were carried out on a cell line generated from NIH-3T3 fibroblast cells which constitutively express the oncogene c-Src (NIH3T3-src) and which form invadosome rosettes [7]. Moreover, these cells constitutively express the yeast peptide Lifeact [8] coupled to a fluorophore, namely mCherry (FIG. 2D). This peptide has the capacity to bind specifically to actin but only in the filamentous form thereof.

Analysis by mass spectrometry of a very small amount of material pushes spectrometers to the limits of their sensitivity. In this context, a majority of the proteins identified actually result from contaminations originating from handling and ambient air. In order to discriminate between the proteins of the microdissected rosettes and the external contaminating elements (ambient air, solutions, etc.), metabolic isotopic labeling of the proteins of interest (C¹³Arginine (Arg⁶) and C¹³Lysine (Lys⁶)) was first performed according to the SILAC method (Stable Isotopic Labeling by Amino acids in Cell culture). This method consists in using a culture medium devoid of arginine and lysine, in which arginine and lysine labeled with carbon C¹³, and unlabeled proline to prevent metabolization of the labeled arginine to labeled proline, are added, and in incorporating this labeling for at least 6 cycles of cell doubling.

The cells expressing the peptide Lifeact coupled to the fluorophore mCherry (FIG. 2D) and labeled with the lysine and arginine isotopes were cultured on a silicone membrane ring (which is covered with gelatin in order to promote adhesion of the cells) placed in a lumox dish 50 (Zeiss).

After adhesion and rinsing with PBS, the cells were fixed with a crosslinking agent, paraformaldehyde (PFA) at 4% in a solution of PBS for 20 minutes. After two rinsing operations, the cells were kept in a solution of PBS at 4° C.

These cells were then labeled with DAPI which is a fluorescent DNA intercalator which makes it possible to visualize the nuclei.

The cells were then placed in a laser microdissector fitted with a dry 63× objective (Zeiss), and the cells are kept in a thin film of PBS.

The rosettes to be cut out were outlined by ways of a stylus on a graphics tablet (dotted circles) before microdissection (FIG. 2E). For the laser dissection, a Zeiss microscope (PALM MicroBeam) was used.

The rosettes were then collected in the cap of a support (collector tube) made of silicone (FIG. 2H).

The support was then washed with a 50 mM Tris-HCl solution, pH 6.8, 7.5% SDS, 20% glycerol, 5% beta-mercaptoethanol, 0.1% bromophenol blue for 2 hours at 95° C., which made it possible to extract the proteins and reverse the fixation thereof.

This extract was then loaded into a well of 10% SDS-PAGE gel and placed under a voltage of 100 volts until the bromophenol blue migrated to the limit between the stacking gel and the separating gel, using a molecular weight marker as a visual control in another well.

A square of gel was then cut out between the upper limit of the well and the migration front was treated for reduction/alkylation of the proteins then proteolysis by trypsin and extraction of the peptides resulting from this digestion.

The peptides were then analyzed by LC-MS/MS with a C18 chromatography gradient for 2 hours and analysis on a mass spectrometer of Q-Exactive (Thermo) type.

The databases were consulted with two different algorithms (Mascot and Sequest) using the Proteome Discoverer software, including the C¹³Arg and C¹³Lys labeling as variable modifications. Only the peptides with high scores, labeled and/or identified with one and/or the other of the two algorithms, were retained.

Table 1 represents the summary of the number of proteins identified during the different experiments. Increasing the number of pieces microdissected firstly made it possible to increase the number of proteins identified, to reach 101 proteins identified after cutting out 10 000 rosettes.

TABLE 1

| | | | Percentage of proteins |
|---|---|---|---|
| | Number of | Number of | strictly identical |
| | pieces | proteins | relative to the |
| Experiment | microdissected | identified | preceding experiment |
| 1 | 350 | 9 | |
| 2 | 3000 | 55 | 44% |
| 3 | 10 000 | 101 | 60% + 20% (proteins |
| | | | very close) = 80% |

It is also observed that, by increasing the amount of material, more peptides are identified corresponding to the proteins identified during the preceding experiment performed with less material. Thus, between experiment 2 and experiment 3, the identification of 60% of the proteins is confirmed and a further 20% of very similar proteins are identified (isoforms, different subunits of the same protein), listed in the following table 2. These elements demonstrate the robustness and reproducibility of this technique.

TABLE 2
Proteins from the 1st experiment found again in experiments 2 and 3
Vimentin OS = Mus musculus GN = Vim
Actin, cytoplasmic 1 (Fragment) OS = Mus musculus GN = Actb
Histone H4 OS = Mus musculus GN = Hist1h4a
Tubulin alpha-1C chain OS = Mus musculus GN = Tuba1c
Common proteins between experiments 2 and 3
Peroxiredoxin-1 (Fragment) OS = Mus musculus GN = Prdx1
40S ribosomal protein S3 OS = Mus musculus GN = Rps3
ATP synthase subunit alpha OS = Mus musculus GN = Atp5a1
Histone H3 (Fragment) OS = Mus musculus GN = H3f3a
Protein Ahnak OS = Mus musculus GN = Ahnak
Histone H2A OS = Mus musculus GN = Hist1h2al
Heterogeneous nuclear ribonucleoprotein H OS = Mus musculus
GN = Hnrnph1
Fructose-bisphosphate aldolase A OS = Mus musculus GN = Aldoa
Tubulin alpha-1B chain OS = Mus musculus GN = Tuba1b
Elongation factor 1-alpha 1 OS = Mus musculus GN = Eef1a1
Histone H2B type 1-F/J/L OS = Mus musculus GN = Hist1h2bf
Heat shock protein HSP 90-beta OS = Mus musculus GN = Hsp90ab1
40S ribosomal protein SA OS = Mus musculus GN = Rpsa
Glyceraldehyde-3-phosphate dehydrogenase OS = Mus musculus GN =
Gapdh
Vimentin OS = Mus musculus GN = Vim
Stress-70 protein, mitochondrial OS = Mus musculus GN = Hspa9
Isoform C of Prelamin-A/C OS = Mus musculus GN = Lmna
Heterogeneous nuclear ribonucleoprotein A1 OS = Mus musculus
GN = Hnrnpa1
Pyruvate kinase PKM OS = Mus musculus GN = Pkm
ATP synthase subunit beta, mitochondrial OS = Mus musculus GN = Atp5b
Elongation factor 2 OS = Mus musculus GN = Eef2
Poly(rC)-binding protein 1 OS = Mus musculus GN = Pcbp1
Actin, cytoplasmic 1 OS = Mus musculus GN = Actb
Histone H4 OS = Mus musculus GN = Hist1h4a
Isoform 2 of 60 kDa heat shock protein, mitochondrial OS =
Mus musculus GN = Hspd1
Tubulin alpha-1A chain OS = Mus musculus GN = Tuba1a
Heat shock cognate 71 kDa protein OS = Mus musculus GN = Hspa8
Polyubiquitin-B (Fragment) OS = Mus musculus GN = Ubb
Histone H2B type 2-E OS = Mus musculus GN = Hist2h2be
Cytoskeleton-associated protein 4 OS = Mus musculus GN = Ckap4
Myosin-9 OS = Mus musculus GN = Myh9
Nucleophosmin OS = Mus musculus GN = Npm1
Phosphoglycerate kinase OS = Mus musculus GN = Pgk1
Proteins from experiment 2 which strongly resemble the proteins detected
during experiment 3
60S ribosomal protein L31 OS = Mus musculus GN = Rpl31
Elongation factor 1-delta (Fragment) OS = Mus musculus GN = Eef1d
T-complex protein 1 subunit gamma OS = Mus musculus GN = Cct3
Heterogeneous nuclear ribonucleoprotein U, isoform CRA_b OS = Mus
musculus GN = Gm28062
Annexin A2 OS = Mus musculus GN = Anxa2
60S ribosomal protein L13 OS = Mus musculus GN = Rpl13
Alpha-actinin-4 OS = Mus musculus GN = Actn4
40S ribosomal protein S8 OS = Mus musculus GN = Rps8
Probable ATP-dependent RNA helicase DDX5 OS = Mus musculus
GN = Ddx5
Elongation factor 1-gamma OS = Mus musculus GN = Eef1g
60S acidic ribosomal protein P0 (Fragment)OS = Mus musculus GN = Rplp0

### Example 2: Validation of the Enrichment

The experiment aimed to demonstrate that the process of the invention (isotopic labeling+targeted laser microdissection+mass spectrometry) makes it possible to enrich the sample with the proteins specifically expressed by the subcellular compartment of interest (i.e. the rosettes).

A range of amounts of proteins from a total cell lyzate was used as a point of comparison, to reach the same amount as that collected with the 40 000 rosettes, and be able to serve as reference (72 ng).

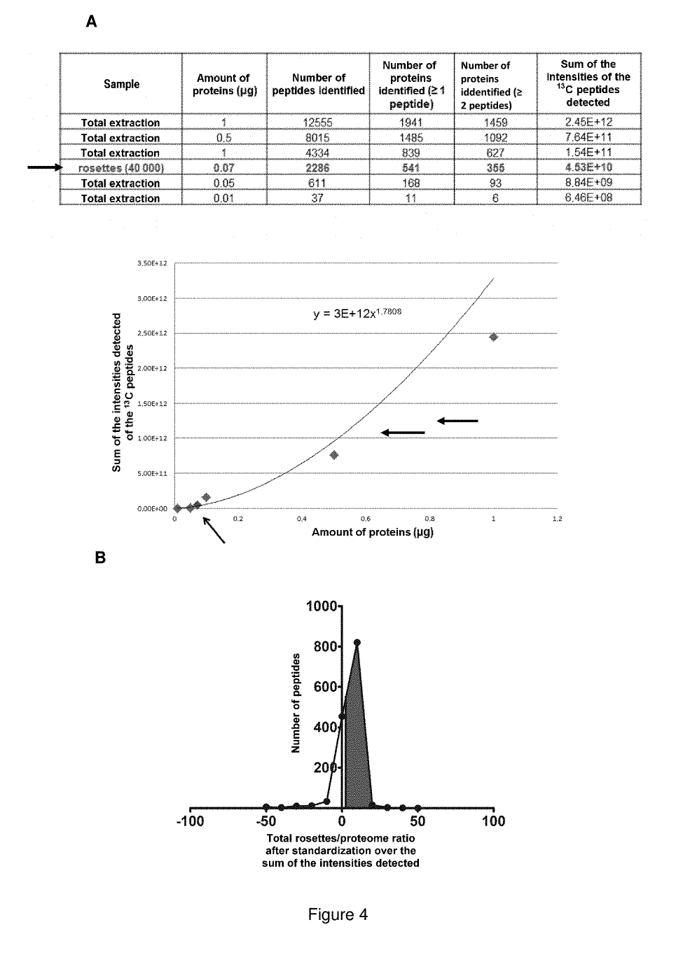

The results are presented in FIG. 4.

### Example 3: Validation by Labeling of the Presence of the Proteins Identified (e.g. Elongation Factor 1-α1)

The NIH3T3-src cells were seeded onto a glass slide then transfected with the HA-eEF1A1 plasmid (provided by Dr. IRWIN MS, University of Toronto, Ontario, Canada) using a transfection agent, lipofectamine 2000 (thermo-Fisher), according to the protocol described by the manufacturer. The cells were then rinsed after 6 hours of incubation, and left to rest for 48 h. The cells were then fixed by the addition of a 4% paraformaldehyde-PBS solution for 10 minutes.

The immunofluorescence protocol was then carried out; the cells were permeabilized by a Triton solution. Finally, the cells were incubated with the primary antibody anti-HA (3F10, Roche) in a primary antibody-PBS-BSA solution, then after washing operations with a solution containing a secondary antibody coupled to a fluorophore before observation with a confocal microscope (Leica SP5).

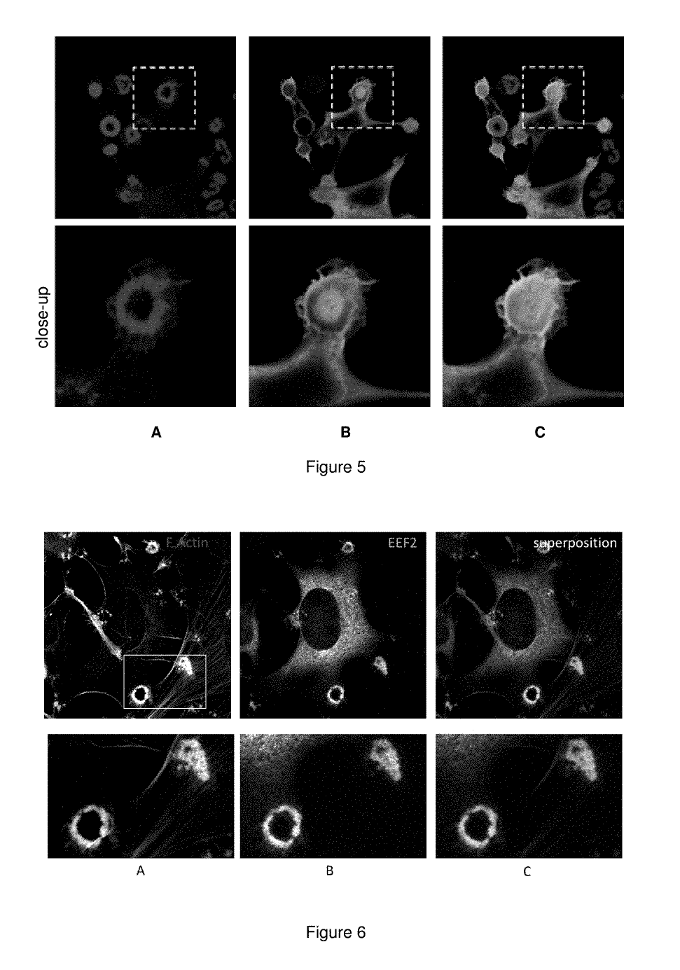

The results are presented in FIG. 5.

### Example 4: Validation by Labeling of the Presence of the Proteins Identified (e.g. HA-Eif2A)

The NIH3T3-src cells were seeded onto a glass slide then transfected with the Flag-EEF2 plasmid (provided by Dr. Huang Y S, Academia Sinica, Taipei, Taiwan) using a transfection agent, lipofectamine 2000 (thermo-Fisher), according to the protocol described by the manufacturer. The cells were then rinsed after 6 hours of incubation, and left to rest for 48 h. The cells were then fixed by the addition of a 4% paraformaldehyde-PBS solution for 10 minutes.

The immunofluorescence protocol was then carried out; the cells were permeabilized by a Triton solution. Finally, the cells were labeled with an anti-flag antibody (Sigma F1804) in an anti-flag antibody-PBS-BSA solution, then after washing operations with a solution containing a secondary antibody coupled to a fluorophore before observation with a confocal microscope (Leica SP5).

The results are presented in FIG. 6.

These experiments, described in examples 3 and 4, made it possible to demonstrate the presence of these proteins, eEF1A1 and HA-Eif2A, in the rosettes, and thereby to validate the procedure for identifying new markers in the rosettes.

Similarly, experiments were carried out which validate the presence of the proteins EEF2, EIF3H, eif4E and Reptin in the rosettes.

### REFERENCE LIST

1. Drissi, R., M. L. Dubois, and F. M. Boisvert, Proteomics methods for subcellular proteome analysis. FEBS J, 2013. 280(22): p. 5626-34.
2. Havrylov, S. and M. Park, MS/MS-based strategies for proteomic profiling of invasive cell structures. Proteomics, 2015. 15(2-3): p. 272-86.
3. Gessel, M. M., J. L. Norris, and R. M. Caprioli, MALDI imaging mass spectrometry: spatial molecular analysis to enable a new age of discovery. J Proteomics, 2014. 107: p. 71-82.
4. Gregorich, Z. R. and Y. Ge, Top-down proteomics in health and disease: challenges and opportunities. Proteomics, 2014. 14(10): p. 1195-210.
5. Ye, H., et al., Top-down proteomics with mass spectrometry imaging: a pilot study towards discovery of biomarkers for neurodevelopmental disorders. PLoS One, 2014. 9(4): p. e92831.
6. Ait-Belkacem, R., et al., MALDI imaging and in-source decay for top-down characterization of glioblastoma. Proteomics, 2014. 14(10): p. 1290-301.
7. Abram, C. L., et al., The adaptor protein fish associates with members of the ADAMs family and localizes to podosomes of Src-transformed cells. J Biol Chem, 2003. 278(19): p. 16844-51.
8. Riedl, J., et al., Lifeact: a versatile marker to visualize F-actin. Nat Methods, 2008. 5(7): p. 605-7.
9. Patent application EP1601450.
10. Leverenz et al., Proteomic Identification of Novel Proteins in Cortical Lewy Bodies. Brain Pathol., 2007. 17(2): p. 139-145.
11. Patent application US2011/0215233.

1. A process for the subcellular proteomic analysis of a test biological sample, comprising:
a) metabolic isotopic labeling of the proteins from a test biological sample;
b) fixation of the sample;
c) labeling of the test subcellular compartment;
d) laser microdissection of the subcellular compartment;
e) extracting the proteins from the subcellular compartment, reversion of the fixation, and proteolysis;
f) analyzing the peptides resulting from step e) by mass spectrometry; and
g) identifying the peptides analyzed.

2. The process as claimed in claim 1, wherein the fixation is performed with a crosslinking agent.

3. The process as claimed in claim 2, wherein the crosslinking agent is paraformaldehyde.

4. The process as claimed in claim 1, wherein the labeling of the subcellular compartment is performed with a fluorescent marker.

5. The process as claimed in claim 1, wherein the subcellular compartment is chosen from a group consisting of the nucleus, cytoplasmic membrane, nuclear membrane, vesicles, mitochondria, lysosome, centriole, proteasome, focal adhesions, lamellipodium, filopodia, invadosome rosette, endoplasmic reticulum, Golgi apparatus, and cell-cell junctions.

6. A process for the in vitro identification of a protein from a biological sample from a subject, comprising:
a) metabolic isotopic labeling of the proteins from a biological sample from a subject;
b) fixation of the sample;
c) labeling of the test subcellular compartment;
d) laser microdissection of the subcellular compartment;
e) extracting the proteins from the subcellular compartment, reversion of the fixation, and proteolysis;
f) analyzing the peptides resulting from step e) by mass spectrometry;
g) identifying of the peptides analyzed;
h) qualitative and/or quantitative comparison of the peptides resulting from step g) relative to the peptides present in a reference sample or to a reference value.

7. The process as claimed in claim 6, wherein the subcellular compartment is the invadosome rosette.

8. The process as claimed in claim 1, wherein the isotope labeling is performed by the SILAC method.

9. The process as claimed in claim 2, wherein the subcellular compartment is chosen from a group consisting of the nucleus, cytoplasmic membrane, nuclear membrane, vesicles, mitochondria, lysosome, centriole, proteasome, focal adhesions, lamellipodium, filopodia, invadosome rosette, endoplasmic reticulum, Golgi apparatus, and cell-cell junctions.

10. The process as claimed in claim 3, wherein the subcellular compartment is chosen from a group consisting of the nucleus, cytoplasmic membrane, nuclear membrane, vesicles, mitochondria, lysosome, centriole, proteasome, focal adhesions, lamellipodium, filopodia, invadosome rosette, endoplasmic reticulum, Golgi apparatus, and cell-cell junctions.

11. The process as claimed in claim 4, wherein the subcellular compartment is chosen from a group consisting of the nucleus, cytoplasmic membrane, nuclear membrane, vesicles, mitochondria, lysosome, centriole, proteasome, focal adhesions, lamellipodium, filopodia, invadosome rosette, endoplasmic reticulum, Golgi apparatus, and cell-cell junctions.

12. The process as claimed in claim 2, wherein the isotope labeling is performed by the SILAC method.

13. The process as claimed in claim 3, wherein the isotope labeling is performed by the SILAC method.

14. The process as claimed in claim 4, wherein the isotope labeling is performed by the SILAC method.

15. The process as claimed in claim 5, wherein the isotope labeling is performed by the SILAC method.

16. The process as claimed in claim 6, wherein the isotope labeling is performed by the SILAC method.

17. The process as claimed in claim 7, wherein the isotope labeling is performed by the SILAC method.
